(19) United States
(12) Patent Application Publication
(10) Pub. No.: US 2004/0253299 A1
(43) Pub. Date: Dec. 16, 2004

(54) Transdermal therapeutic system (reservoir-tts) for using pramipexole and ropinirole

(76) Inventors: Cornelia Beier, Heidenheim (DE); Martina Wilhelm, Holzkirchen (DE)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/485,043

(22) PCT Filed: Jul. 26, 2002
(86) PCT No.: PCT/EP02/08393

(30) Foreign Application Priority Data
Jul. 30, 2001 (DE) 101 37 162.4

(51) Int. Cl. A61K 9/70

(52) U.S. Cl. 424/449

(57) ABSTRACT

The invention relates to an active-ingredient-containing, transdermal therapeutic system having a reservoir for the administration of pramipexole, ropinirole, pharmaceutically acceptable salts thereof or pharmaceutically acceptable derivatives thereof.

The invention relates to an active-ingredient-containing transdermal therapeutic system having a reservoir for the administration of pramipexole, ropinirole, pharmaceutically acceptable salts thereof or pharmaceutically acceptable derivatives thereof.

Pramipexole [2-amino-6-n-propylamino-4,5,6,7-tetrahydrobenzothiazole] is used especially for the treatment of Parkinson's disease. As a dopamine agonist, it binds with high selectivity and specificity to the D₂ and D₃ receptors. Owing to the stimulation of the dopamine receptors in the corpus striatum, pramipexole produces a reduction in the motor disturbances that occur in Parkinson's disease. When administered orally, the daily dose of pramipexole is from 1.5 to 4.5 mg. The bioavailability is 90%. However, the administration of even small amounts of pramipexole is associated with considerable side-effects in the patient.

Ropinirole [4-(2-di-n-propylaminoethyl)-2-(3H)-indolone], as a selective dopamine agonist acting on the D₂ receptors, is also used for the treatment of Parkinson's disease. The daily dose when administered orally is from 0.3 to 30 mg. The bioavailability is 50%.

By means of a transdermal therapeutic system it is possible to circumvent the side-effects that occur in the case of oral administration of pramipexole or ropinirole. Transdermal administration furthermore has the advantage that the active ingredient, after permeation through the skin, has a direct systemic action, as a result of which a constant blood plasma level can be guaranteed. Hepatic metabolism of the active ingredient is also circumvented, that is to say, the burden on the liver is relieved. Gastrointestinal side-effects are avoided. The use of patches that retain their full effectiveness for several days, which in contrast to oral administration is simple and convenient, is an advantage for the patient. Since the system is applied externally, it can fulfil its intended function for a very long time without being changed.

There is already known from EP-B1-0 428 038 a transdermal system having a content of pramipexole and

a) an active-ingredient-impermeable backing layer which is at the same time constructed as a covering plaster,

b) an active-ingredient-containing reservoir (preferred carrier for the active ingredient is an emulsion-polymerised polyacrylate of the type Eudragit NE 30 D^{R} produced by Röhm GmbH, Darmstadt) and

c) a peel-off protective film (release liner).

Owing to the surfactants used in an emulsion-stabilised polyacrylate, a TTS produced according to EP-B1-0 428 038 does not exhibit sufficient stability of the active ingredient. In that matrix, pramipexole decomposes very rapidly, with discoloration occurring. In addition, the active ingredient crystallises out. That patch does not, therefore, have sufficient stability in storage.

For a matrix-controlled transdermal therapeutic system having a content of pramipexole and polyacrylate(s) as self-adhesive matrix material(s) it has been found that 90% of the active ingredient is released within 24 hours irrespective of the amount used. That is to say, the period for which that patch is worn would be only one day.

WO 99/49853 proposes a moisture-activatable transdermal therapeutic system in which ropinirole hydrochloride is incorporated in a matrix together with an activator that gives a basic reaction in water, such as, for example, hydrated sodium silicate. The unstable ropinirole base, which has good permeation properties, is released from the stable ropinirole hydrochloride, which has a poor permeation capacity, only on the skin as a result of the admission of moisture. The release of the active ingredient and hence also the permeation of the active ingredient is therefore dependent on the moisture of the skin, which may possibly lead to irregular permeation rates and hence to fluctuating blood levels.

Reservoir-TTSs having a content of ropinirole are described in WO 96/39136 and WO 97/11696.

According to WO 96/39136, a reservoir comprising ropinirole base, salt/propylene glycol (1:1), a membrane of ethylene-vinyl acetate copolymer and a silicone adhesive layer are used for a patch. For a 30 cm² patch, in vitro permeation through human skin gives a daily dose of 300 μg.

WO 97/11696 describes a patch consisting of a cover layer (polyester), a silicone adhesive layer, a reservoir (solution of ropinirole hydrochloride, oleic acid/polyethylene glycol or polyethylene glycol monolaurate/polyethylene glycol in a super-absorbent material), a silicone adhesive layer and a peel-off film.

The object of the present invention is to provide a reservoir-TTS having a content of pramipexole, ropinirole, pharmaceutically acceptable salts thereof or pharmaceutically acceptable derivatives thereof, the stability of which with regard to the break-down of the active ingredient meets the standards required for authorisation, the total amount of active ingredient being intended to be released over a period of at least 3 days.

Surprisingly, it has now been found that a reservoir-TTS comprising pramipexole, ropinirole, salts thereof or derivatives thereof, where applicable with the addition of chelate formers or antioxidants as stabilisers, is to a large extent stable towards decomposition and exhibits an active ingredient release over a period of 3 days or more.

A reservoir-TTS according to the invention consists of an impermeable cover layer, an active-ingredient-containing reservoir or a reservoir layer, a semi-permeable membrane, an optional pressure-sensitive adhesive layer and a peel-off protective layer.

There come into consideration as the impermeable cover layer films that are acetal, acrylate, acrylonitrile-butadiene-styrene, acrylonitrile (methyl methacrylate) copolymer, acrylonitrile copolymer, ethylene ethyl acrylate, ethylene methyl acrylate, ethylene vinyl acetate, ethylene vinyl acetate copolymer, ethylene vinyl alcohol copolymer, ionomer, Nylon (polyamide), Nylon (polyamide) copolymer, polybutylene, polycarbonate, polyester, polyethylene terephthalate, thermoplastic polyester copolymer, polyethylene copolymer (high-density), polyethylene (high-molecular-weight, high-density), polyethylene (intermediate-molecular-weight, high-density), polyethylene (linear, low-density), polyethylene (low-density), polyethylene (medium-density), polyethylene oxide, polyimide, polypropylene, polypropylene (coated), polypropylene (oriented), polystyrene, polyurethane, polyvinyl acetate, polyvinyl chloride, polyvinylidene chloride and/or styrene-acrylonitrile films, which may, if required, be metallised or pigmented.

For the peel-off protective layer there come into consideration polyethylene terephthalate, polyester, polyethylene, polypropylene, polysiloxane, ethylene vinyl acetate, polyurethane or paper or a mixture thereof, usually with a silicone, fluorosilicone or fluorocarbon coating.

The reservoir comprises pramipexole, ropinirole, pharmaceutically acceptable salts thereof or pharmaceutically acceptable derivatives thereof, and one or more solvents in which the active ingredient, active ingredient carriers, permeation enhancers, solubilisers, stabilisers, emulsifiers, preservatives, thickeners and/or customary membrane system or reservoir patch adjuvants are dissolved. The reservoir or the reservoir layer is formed by the cover layer (carrier film) and the membrane. The active ingredient carrier (=solvent) may be a low-molecular-weight monohydric alcohol, such as ethanol, 1-propanol or isopropanol, a low-molecular-weight polyhydric alcohol, for example propylene glycol, or an ester, such as isopropyl myristate, or may be a mixture thereof, where applicable also in the form of an aqueous mixture.

Pharmaceutically acceptable salts of pramipexole or ropinirole are understood as being acid addition salts. The latter are obtained by reaction of the active ingredient in the form of the free base with pharmaceutically acceptable acids. Pharmaceutically acceptable acids are inorganic acids (for example hydrochloric acid, hydrobromic acid, sulfuric acid, nitric acid and phosphoric acid) or organic acids (for example acetic acid, propionic acid, hydroxyacetic acid, lactic acid, pyruvic acid, oxalic acid, maleic acid, malonic acid, succinic acid, fumaric acid, malic acid, tartaric acid, citric acid, methanesulfonic acid, ethane-sulfonic acid, benzenesulfonic acid, p-toluenesulfonic acid, cyclohexanesulfamic acid, salicylic acid, p-aminosalicylic acid and pamoic acid). Solvates with the active ingredient are also referred to as acid addition salts. Such solvates are, for example, hydrates and alcoholates.

The amount of pramipexole, ropinirole, salt thereof or derivative thereof used in the transdermal therapeutic system according to the invention ranges from 2 to 30% by weight in the reservoir or in the solution (filling solution) with which the reservoir is filled.

As stabilisers it is possible to use antioxidants, such as vitamin E, butylhydroxytoluene, butylhydroxyanisole, ascorbic acid and/or ascorbyl palmitate, and/or chelate formers, such as disodium ethylenediaminetetraacetic acid, potassium citrate and/or sodium citrate.

The membrane, which usually consists of inert polymers, especially those based on polyethylene, polypropylene, polyvinyl acetate, polyamide, ethylene-vinyl acetate copolymers and/or silicone, may, depending on the pore size, have a controlling effect on the release of the active ingredient. Preferably, polyethylene is used.

For the pressure-sensitive adhesive layer it is possible to select a pressure-sensitive adhesive based, for example, on polyurethane, polyisobutylene, polyvinyl ether, silicone, polyacrylate or a mixture thereof, for example a polyacrylate pressure-sensitive adhesive.

The adhesive based on silicone may be formed by silicone adhesives that are based on two main components: a polymer, especially polydimethylsiloxane or polydimethyldiphenylsiloxane, and a resin having a three-dimensional silicate structure. In the production of a silicone adhesive, a condensation reaction takes place between polymer chains and resin, since both have terminal silanol groups. The mixing ratio of resin to polymer and the degree of silanol functionality determine the physical properties of silicone adhesives; cf., for example, Sobieski et al., “Silicone Pressure Sensitive Adhesives”, Handbook of Pressure Sensitive Adhesive Technology, 2nd edition, pages 508-517 (D. Satas, ed.); Van Nostrand Reinhold, New sive Technology, 2nd edition, pages 508-517 (D. Satas, ed.), Van Nostrand Reinhold, New York (1989).

A further example of a pressure-sensitive adhesive based on silicone is trimethylated silicon dioxide that has been treated with polydimethylsiloxane having terminal trimethylsilyloxy groups.

The adhesives based on acrylate may be any desired homopolymer, copolymer or ter-polymer consisting of various acrylic acid derivatives.

For example, the polyacrylates may be polymers of one or more monomers of acrylic acid and other copolymerisable monomers. In addition, the acrylate polymers may include copolymers of alkyl acrylates and/or alkyl methacrylates and/or copolymerisable secondary monomers or monomers having functional groups. If the amount of any type added as monomer is altered, the cohesive properties of the resulting acrylate polymers can be altered. In general, the acrylate polymer consists of at least 50% by weight of an acrylate, methacrylate, alkyl acrylate or alkyl methacrylate monomer, from 0 to 20% of a functional monomer copolymerisable with acrylate, and from 0 to 50% of another monomer.

Various acrylate monomers are mentioned hereinafter, such as, for example, acrylic acid, methacrylic acid, butyl acrylate, butyl methacrylate, hexyl acrylate, hexyl methacrylate, isooctyl acrylate, isooctyl methacrylate, glycidyl methacrylate, 2-hydroxyethyl acrylate, methyl acrylate, methyl methacrylate, 2-ethylhexyl acrylate and 2-ethylhexyl methacrylate, that may be polymerised individually or in admixture.

In addition, functional monomers that are copolymerisable with the above-mentioned acrylates, such as, for example, acrylic acid, methacrylic acid, hydroxyethyl acrylate, hydroxypropyl acrylate, acrylamide, dimethylacrylamide, acrylonitrile, dimethylaminoethyl acrylate, dimethylaminoethyl methacrylate, tert.-butylaminoethyl acrylate, tert.-butylaminoethyl methacrylate, methoxyethyl acrylate, vinyl acetate and methoxyethyl methacrylate, can be used for copolymerisation.

The pressure-sensitive adhesives, especially those based on acrylate or silicone, may be alcohol-resistant, for example an alcohol-resistant polyacrylate pressure-sensitive adhesive.

The pressure-sensitive adhesive layer may be applied to the membrane in such a manner that it covers the entire surface area or an annular portion thereof.

As permeation enhancers and/or solubilisers it is possible to use monohydric and/or polyhydric aliphatic, cycloaliphatic and/or aromatic-aliphatic alcohols each having up to eight carbon atoms, for example ethanol, 1,2-propanediol, dexpanthenol and/or polyethylene glycol; alcohol/water mixtures; saturated and/or unsaturated fatty alcohols each having from 8 to 18 carbon atoms; terpenes, for example cineol, carveol, menthone, trepineol, verbenone, menthol, limonene, thymol, cymene, terpinene4-ol, neomenthol, geraniol and/or fenchone: mixtures of terpenes and ethanol and/or propylene glycol; tea tree oil; saturated and/or unsaturated cyclic ketones; alkyl methyl sulfoxides; saturated and/or unsaturated fatty acids each having from 8 to 18 carbon atoms; esters and salts thereof; natural vitamin E (Copherol® F1300); synthetic vitamin E and/or vitamin E derivatives; sorbitan fatty acid esters and ethoxylated sorbitan fatty acid esters; azones (laurocapram); azones mixed with alcohols; urea; 1-alkylpyrrolidone; polyvinylpyrrolidone; block copolymers of polyethylene glycol and dimethylsiloxane with a cationic group at one end; polysiloxanes; folate-polyethylene glycol liposome, proliposome; phospholipids; polyoxyethylene-10-stearyl ether; mixture of polyoxyethylene-10-stearyl ether and glyceryl dilaurate; dodecyl-2-(N,N-dimethylamino)-propanol tetradecanoate and/or dodecyl-2-(N,N-dimethylamino)-propionate; N-acetylprolinate esters with >8 carbon atoms; non-ionic surfactants, for example lauryl ethers and/or esters of polyoxyethylene; ethosome; (phospholipid vesicle); dimethyl(arylimino)sulfuran; mixture of oleic acid analogues and propylene glycol; mixture of padimate O, octyl salicylate, octylmethoxy cinnamate and laurocapram or a mixture of individual components; isopropyl myristate, isopropyl palmitate or water or a mixture of individual components.

The invention is described in more detail by the following Examples without, however, thereby limiting the scope of the invention.

### EXAMPLE 1

Production of a Reservoir-TTS According to the Invention with Pramipexole

A pressure-sensitive adhesive based on polyacrylate (Durotak 87-4098) was used for the adhesive layer. A peel-off layer of PET was coated with the polyacrylate adhesive by means of a coating system. A microporous polyethylene membrane (DSM Solupor 10P05A) was laminated to the coated peel-off film, so that a laminate consisting of peel-off film, adhesive and membrane was produced. The laminate was then welded by means of a sealing machine (having a welding ring) to a carrier film of polyester (aluminized, with a polyolefin sealing layer (heat-sealable)) in such a manner that a gap remained for introduction of an active ingredient solution. The fillable transdermal therapeutic system (empty TTS) was filled, for example using a Hamilton syringe or using a hose pump with a cannula, with the following active ingredient solution (2 ml). After filling, the filling gap was welded. Filled transdermal therapeutic systems were punched out by means of a punch.

Composition of the active ingredient solution in the TTS:

| | |
|---|---|
| pramipexole: | 4.5 g |
| ethanol abs.: | 15.3 g |
| propylene glycol: | 4.59 g |
| Copherol F1300: | 5.36 g |
| Klucel HF: | 0.25 g |
| total | 30 g |

Determination of the Permeation of Pramipexole In Vitro through Mouse Skin

Apparatus for the skin permeation:

| | |
|---|---|
| cells: | modified flow cell |
| skin: | hairless mouse from female mice |
| acceptor medium: | 0.9% sodium chloride + 0.05% sodium azide, |
| | 60 ml per cell |
| permeation temp.: | 32° C. ± 0.5° C. |

After taking samples, the active ingredient concentrations are then determined by means of HPLC.

| time | permeation |
|---|---|
| [h] | [mg/cm²] |
| 24 | 8.44 |
| 144 | 24.7 |

### EXAMPLE 2

Production of a Reservoir-TTS According to the Invention with Pramipexole Dihydrochloride

The reservoir-TTS is produced from a carrier film, a microporous polyethylene membrane (DSM Solupor 10P05A), an annular adhesive layer and a peel-off film of PET. The reservoir lies between the carrier film and the membrane. For that purpose, the membrane is welded by means of a sealing machine (having a welding ring) to a carrier film of polyester (aluminized, with a polyolefin sealing layer (heat-sealable)) in such a manner that a gap remains for introduction of an active ingredient solution. The fillable transdermal therapeutic system (empty TTS) was filled, for example using a Hamilton syringe or using a hose pump with a cannula, with the following active ingredient solution (2 ml). After filling, the filling gap was welded. Filled transdermal therapeutic systems were punched out by means of a punch. For affixing the system, an annular adhesive layer provided with a peel-off film is laminated to the membrane.

Composition of the active ingredient solution in the TTS:

| | |
|---|---|
| pramipexole dihydrochloride | 28 mg |
| Copherol | 175 mg |
| propylene glycol | 311 mg |
| ethanol abs. | 486 mg |

Determination of the Permeation of Ropinirole In Vitro through Mouse Skin

Apparatus for the skin permeation:

| | |
|---|---|
| cells: | modified flow cell |
| skin: | hairless mouse from female mice |
| acceptor medium: | 0.9% sodium chloride + 0.05% sodium azide, |
| | 60 ml per cell |
| permeation temp.: | 32° C. ± 0.5° C. |

After taking samples, the active ingredient concentrations are then determined by means of HPLC.

| time | permeation |
|---|---|
| [h] | [mg/cm²] |
| 24 | 300 |
| 48 | 825 |

### EXAMPLE 3

Production of a Reservoir-TTS According to the Invention with Ropinirole

The reservoir-TTS is produced from a carrier film, a microporous polyethylene membrane (DSM Solupor 10P05A), an annular adhesive layer and a peel-off film of PET. The reservoir lies between the carrier film and the membrane. For that purpose, the membrane is welded by means of a sealing machine (having a welding ring) to a carrier film of polyester (aluminized, with a polyolefin sealing layer (heat-sealable)) in such a manner that a gap remains for introduction of an active ingredient solution. The fillable transdermal therapeutic system (empty TTS) was filled, for example using a Hamilton syringe or using a hose pump with a cannula, with the following active ingredient solution (2 ml). After filling, the filling gap was welded. Filled transdermal therapeutic systems were punched out by means of a punch. For affixing the system, an annular adhesive layer provided with a peel-off film is laminated to the membrane.

Composition of the active ingredient solution in the TTS:

| | |
|---|---|
| ropinirole: | saturated solution |
| isopropyl myristate: | 18% by weight |
| propylene glycol: | 32% by weight |
| ethanol abs.: | 50% by weight |

Determination of the Permeation of Ropinirole In Vitro through Mouse Skin

Apparatus for the skin permeation:

| | |
|---|---|
| cells: | modified flow cell |
| skin: | hairless mouse from female mice |
| acceptor medium: | 0.9% sodium chloride + 0.05% sodium azide, |
| | 60 ml per cell |
| permeation temp.: | 32° C. ± 0.5° C. |

After taking samples, the active ingredient concentrations are then determined by means of HPLC.

| time | permeation |
|---|---|
| [h] | [mg/cm²] |
| 22 | 4.19 |
| 48 | 6.74 |

1. A transdermal therapeutic system (TTS) for the administration of one or more active ingredients selected from the consisting of pramipexole, ropinirole, pharmaceutically acceptable pramipexole salt, and pharmaceutically acceptable ropinirole derivative, the system comprising:
(i) an active-ingredient-impermeable cover layer,
(ii) a reservoir or reservoir layer having a content of active ingredient,
(iii) a semi-permeable membrane that controls the release of the active ingredient,
(iv) an adhesive layer for skin contact of the system, and
(v) a peel-off protective layer,
wherein the reservoir or reservoir layer comprises one or more active ingredient carriers selected from the group consisting of low-molecular-weight monohydric or polyhydric alcohols, esters, and mixtures thereof.

2. The system of claim 1, wherein one or more salts of at least one of pramipexole and ropinirole comprise reaction products of pramipexole, ropinirole, a pramipexole derivative or a ropinirole derivative and an acid.

3. The system of claim 1, wherein the active ingredient is one or more solvates comprising a reaction product of pramipexole, ropinirole, a pramipexole salt, a ropinirole salt, a pramipexole derivative or a ropinirole derivative and a solvate former.

4. The system claim 1, wherein the active ingredient is a solvate salt of an inorganic or organic acid, and wherein the system is free of an acid-neutralizing base.

5. The system of claim 1, wherein the reservoir or reservoir layer is free of inorganic mineral salts.

6. The system of claim 1, wherein the reservoir or reservoir layer comprises one or more members of the group consisting of permeation enhancers, solubilizers, stabilizers, emulsifiers, preservatives, thickeners, membrane system, and reservoir patch adjuvants.

7. The system of claim 6, wherein the stabilizers comprise one or more members of the group consisting of chelate formers and antioxidants.

8. The system of claim 7, wherein the chelate formers comprise one or members more of the group consisting of disodium ethylenediaminetetraacetic acid, potassium citrate, and sodium citrate.

9. The system of claim 7, wherein the antioxidants comprise one or more members of the group consisting of vitamin E, butylhydroxytoluene, butylhydroxyanisole, ascorbic acid, and ascorbyl palmitate.

10. The system of claim 1, wherein the membrane controls the release of the active ingredient.

11. The system of claim 1, wherein the adhesive layer covers the entire surface area of the membrane or an annular portion thereof.

12. The system of claim 1, wherein the adhesive layer is a pressure-sensitive adhesive.

13 (Canceled).

14. The system of claim 1, wherein the active ingredient is selected from the group consisting of pramipexole, pramipexole salt, pramipexole derivative, and mixtures thereof, and the active ingredient carrier is an aqueous mixture of low-molecular-weight monohydric or polyhydric alcohols and esters.

15. The system of claim 1, wherein the monohydric alcohol is ethanol.

16. The system of claim 1, wherein the polyhydric alcohol is propylene glycol.

17. The system of claim 2, wherein the acid is an inorganic acid selected from the group consisting of hydrochloric acid, hydrobromic acid, sulfuric acid, nitric acid, and phosphoric acid.

18. The system of claim 2, wherein the acid is an organic acid selected from the group consisting of carboxylic acids, propionic acid, hydroxyacetic acid, lactic acid, pyruvic acid, oxalic acid, maleic acid, malonic acid, succinic acid, fumaric acid, malic acid, tartaric acid, citric acid, methanesulfonic acid, ethanesulfonic acid, benzenesulfonic acid, p-toluenesulfonic acid, cyclohexanesulfamic acid, salicylic acid, p-aminosalicylic acid and pamoic acid.

19. The system of claim 18, wherein the carboxylic acid is acetic acid.

20. The system of claim 3, wherein the solvate former is water or alcohol.

21. The system of claim 20, wherein the solvate former is an alcohol and the alcohol is ethyl alcohol.

22. The system of claim 12, wherein the adhesive is selected from the group consisting of an acrylate and silicone.

23. The system of claim 22, wherein the acrylate is a polyacrylate pressure-sensitive adhesive.

24. The system of claim 23, wherein the polyacrylate pressure-sensitive adhesive is alcohol-resistant.
